(19) United States
(12) Patent Application Publication
(10) Pub. No.: US 2022/0080478 A1
(43) Pub. Date: Mar. 17, 2022

(54) MANAGEMENT STATION OF MULTI-POINT TIME-SHARING WATER QUALITY MONITORING SYSTEM

(71) Applicant: Shang-Jung WU, Taoyuan City (TW)

(72) Inventor: Shang-Jung WU, Taoyuan City (TW)

(21) Appl. No.: 17/020,019

(22) Filed: Sep. 14, 2020

(51) Int. Cl. B08B 13/00; G01N 33/18; G05D 1/10; B64C 39/02; G05D 1/12; B64F 1/36; B64D 45/00; B08B 1/00; B08B 3/02

(52) U.S. Cl. CPC B08B 13/00; G01N 33/18; G05D 1/101; B64C 39/024; B64C 2201/141; B64F 1/362; B64D 45/00; B08B 1/002; B08B 3/02; G05D 1/12

(57) ABSTRACT

A management station of multi-point time-sharing water quality monitoring system for managing a water quality sensor machine is disclosed to include a landing platform, a main control module, a wireless transmission module, a washing machine and a charging unit. When the unmanned vehicle returns to the landing platform from one sensing point, the main control module automatically controls the washing machine to wash the water quality sensor module, and also controls the charging unit to charge a power battery installed on the unmanned vehicle. The management station can not only manage all online water quality sensor machines, and can also automatically complete washing and charging operations after the unmanned vehicle returns, and then the unmanned vehicle continues to fly to the next sensing point to perform the collection task according to the set flight path, until all tasks are completed. This design streamlines manpower and reduces maintenance costs.

### BACKGROUND OF THE INVENTION

### 1. Field of the Invention

The present invention relates to water quality monitoring technology and more particularly, to a management station of multi-point time-sharing water quality monitoring system, which can control an unmanned vehicle equipped with a water quality sensor module to travel to and from multiple sensing points to perform various water quality parameter data collection tasks. After the unmanned vehicle returns, the management station can automatically charge the unmanned vehicle and wash the water quality sensor module.

### 2. Description of the Related Art

Today, natural seawater, aquaculture ponds or aquariums are used to stock useful aquariums, aquatic products, etc., including fish, shrimp or aquatic plants and other aquatic animals and plants. In addition to aquaculture pond equipment, other water quality, stocking quantity, stocking time, the amount of bait, temperature, etc. are all important factors that affect the aquaculture effect. In order to have better growth conditions for aquaculture, most of the aquaculture ponds are currently equipped with a number of waterwheels, automatic feeders, filters and other aquaculture equipment, and a variety of electronic sensing devices will be used to obtain water quality information in the aquaculture pond. When the water quality of the aquaculture pond changes, it can be immediately sensed and reported to the farmer to take corresponding measures to avoid death due to the setting of water quality conditions and the ecological environment not suitable for the growth and changes of aquatic animals.

However, the organisms stocked in the general aquaculture ponds may come from different environments and require different water quality conditions, and their ecological composition is quite complicated. Therefore, in the aquaculture process, including water quality conditions (such as dissolved oxygen, temperature, pH, salinity, etc.), water pollution (such as ammonia nitrogen, suspended solids, redox potential, etc.) and plankton, free-floating plants, etc., these are the keys that will affect the aquaculture's ability to prevent disease and aquaculture success. Therefore, maintaining good water quality is extremely important for the health of aquatic animals. A common practice is to monitor the acidity and alkalinity (i.e. PH value) and dissolved oxygen content (i.e. DO value) of the water quality in the aquaculture pond. If the pH value of the water quality deviates from the allowable value, the pump will be started for pumping and water replacement. The necessary stabilizers such as lime, humic acid, and probiotics can also be put in to disinfect and decompose the decayed substances at the bottom of the pond and provide algae nutrients, adjust the pH of the water quality and maintain plankton, free-floating plant density, etc. Then, wait for a period of time before testing, until the pH value is within the allowable range. When the amount of dissolved oxygen in the water is insufficient, the waterwheel is started to fetch water or an aerator to replenish the dissolved oxygen in the water, thereby ensuring the balance and continued survival of the ecology in the aquaculture pond.

In the conventional continuous water quality monitoring system, each aquaculture pond in a different area needs a water quality collection device for detection. If it is used in multiple different breeding tanks, in order to achieve unified control of different aquaculture ponds and to monitor water quality parameters (such as water temperature, dissolved oxygen, acidity, salinity, ammonia nitrogen, suspended solids, redox potential, etc.) at any time, you need to have multiple sets of water quality collection devices. Not only is the cost of construction very high, but it also causes farmers to have a low willingness to adopt a continuous water quality monitoring system.

As shown in FIG. 3, it is a block diagram of a conventional multi-point water quality monitoring system. The multi-point water quality monitoring system sets the multiple sensor modules A1 (including the water quality sensor and signal processor, etc.) of the water quality collection device A in different aquaculture ponds, and the signal collection and storage unit A2 will collects the water quality parameter data sensed by the water quality sensors by wired or wireless transmission for storage, and then uploads the data to the background server or cloud processing platform B through the Internet for various monitoring or control. However, the multi-point water quality monitoring system usually places the water quality sensors in the water of the aquaculture ponds for a long time. If the detected water is highly polluted or contains rich plankton and free-floating plants, it will cause optical water quality sensors to be very easy to attach algae, shellfish, etc., so that the sensing signal is quickly attenuated or distorted. Electrochemical water quality sensors are also prone to rapid aging due to the continuous reaction of the electrode. It is necessary to send personnel to regularly go to the aquaculture ponds at different locations for frequent calibration, repair and maintenance, etc., which is extremely dependent on manpower. Once neglected to maintain it will cause a significant impact, and even lead to system failures, misjudgments and other situations.

Therefore, in the era of using automatic water quality monitoring and control in aquaculture ponds, the water quality parameters in closed water will not change frequently or drastically. Therefore, how to design a multi-point time-sharing water quality monitoring system that can simplify manpower in the maintenance of water quality sensor to effectively improve the many inconveniences and deficiencies in the unified control of multiple aquaculture ponds has long been an important issue that the industry is eager to improve.

### SUMMARY OF THE INVENTION

The present invention has been accomplished under the circumstances in view. It is therefore the main object of the present invention to provide a management station of multi-point time-sharing water quality monitoring system, which is used to manage a water quality sensor machine that comprises an unmanned vehicle and a water quality sensor module carried on the unmanned vehicle. The unmanned vehicle is used to receive a task command to travel to and from a number of designated sensing points according to a pre-set flight path and to perform various water quality parameter data collection tasks through the water quality sensor module. The management station comprises a landing platform for the positioning of the unmanned vehicle, a main control module, a wireless transmission module, a washing machine and a charging unit. The main control module is used to manage the operation of the water quality sensor machine and receive the various water quality parameter data through the wireless transmission module. When the unmanned vehicle returns to the landing platform from one sensing point, the main control module automatically controls the washing machine to wash the water quality sensor module, and also controls the charging unit to charge a power battery installed on the unmanned vehicle. The management station can not only manage all online water quality sensor machines, including unmanned vehicle lifting, real-time tracking and recording flight trajectory and movement, etc., and can also automatically complete washing and charging operations after the unmanned vehicle returns, and then the unmanned vehicle continues to fly to the next sensing point to perform the collection task according to the set flight path, until all tasks are completed. This design streamlines manpower and reduces maintenance costs.

It is another object of the present invention to provide a management station of multi-point time-sharing water quality monitoring system, wherein the charging unit comprises a cooling module. The cooling module is preferably a cooling fan. The cooling fan causes currents of air toward the water quality sensor machine or the power battery directly to avoid the high temperature of the power battery during the charging process causing loss or reduced charging performance, and thus extending the overall service life.

It is still another object of the present invention to provide a management station of multi-point time-sharing water quality monitoring system, which further comprises an alarm unit electrically connected to the main control module. When the main control module detects abnormal conditions of the unmanned vehicle, the alarm unit will send out an alarm signal externally, including warning sound, voice, light source or electronic message, to inform the on-site operator to deal with it in real time to ensure normal operation of the unmanned vehicle.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is a system block diagram of the present invention.

FIG. 2 is a block diagram of an alternate form of the management station of multi-point time-sharing water quality monitoring system

FIG. 3 is a block diagram of a conventional multi-point water quality monitoring system

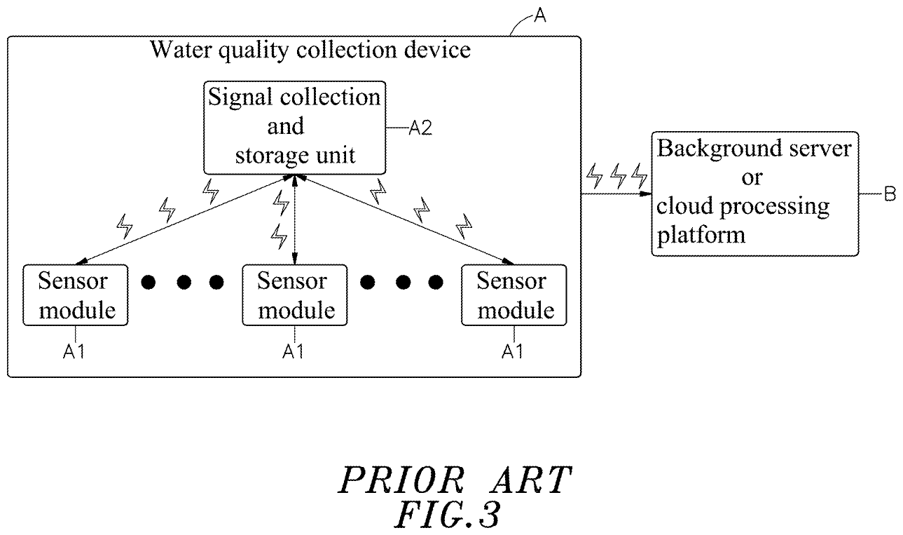

### DETAILED DESCRIPTION OF THE PREFERRED EMBODIMENT

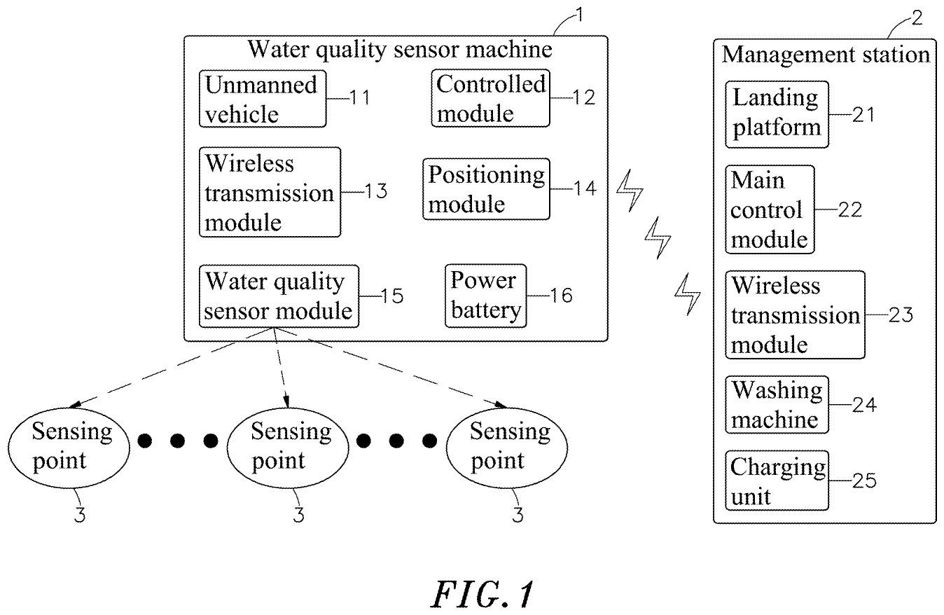

Referring to FIG. 1, a system block diagram of the present invention is shown. As shown, the management station of multi-point time-sharing water quality monitoring system of the present invention is suitable for managing at least one water quality sensor machine 1.

The water quality sensor machine 1 comprises an unmanned vehicle 11, a controlled module 12, a wireless transmission module 13, a positioning module 14, a water quality sensor module 15 and a power battery 16.

The unmanned vehicle 11 is an unmanned aerial vehicle (UAV) or unmanned aircraft system (UAS), also known as aerial camera, remote control drone, etc. A power unit is installed on the frame of the unmanned vehicle 11, and the power unit is mainly composed of a propeller wing and a motor to form a rotorcraft, fixed wing aircraft or helicopter rotor machine. The vehicle body is driven to fly by the reaction force generated when the propeller wing rotates.

On the unmanned vehicle 11, sensors such as gyroscopes, accelerometers, magnetometers, and barometers are generally used as feedback. The unmanned vehicle 11 is controlled by the controlled module 12 (including the controller, signal processing unit and storage unit) to receive the task command or control signal through the wireless transmission module 13. You can cooperate with the positioning module 14 (including global positioning system (GPS) or inertial navigation system) to obtain the attitude, heading, or altitude of the unmanned vehicle 11. It can also lock the unmanned vehicle 11 at a specified position and altitude for positioning and navigation, and can also track and record the flight trajectory and movement of the unmanned vehicle 11 in real time, so that it has attitude stabilization and control, mission equipment management, and emergency control functions. In this embodiment, the water quality sensor module 15 is attached to the bottom of the frame of the unmanned vehicle 11. The water quality sensor module 15 includes at least one water quality sensor, which is used to sense physical parameters including but not limited to water temperature, water pressure, etc., dissolved oxygen, acidity, salinity, ammonia nitrogen (NH3-N), suspended solids, redox potential and other chemical parameters, or other water quality parameters related data. The data to be detected can be any one of the above water quality parameters or a combination of two or more of the above water quality parameters. After the data of various water quality parameters are integrated through the controlled module 12, the integrated data is fed back by the wireless transmission module 13 to a management station 2 or the database of the back-end server for storage by the 3G, 4G-LTE or 5G-NR mobile communication network. In addition, the unmanned vehicle 11 is equipped with the power battery 16, which can be a lithium polymer battery, to provide the overall required power.

In addition, the controlled module 12 of the water quality sensor machine 1 can obtain the task command issued by the management program of the background server or cloud processing platform through the wireless transmission module 13, and automatically plan at least two sets of fixed-point round-trip flight routes. According to the weather information provided by the management program, after determining that the weather conditions are suitable for flight, all online water quality sensor machine 1 will automatically complete each collection task according to the task command issued, and quickly transmit the water quality sensor machine 1 real-time return image and water quality sensing data, etc., so that the field operators can understand the task execution status of the water quality sensor machine 1. You can also operate the flight actions in real time with the audio and video on the streaming machine, set the flight area and verify the flight rights, etc. The management program of the background server connects to the open data platform of the Meteorological Bureau through the application program interface (API) to obtain weather information of each district, including rainfall, wind speed, etc., and can plan and correct the latest flight route according to the real-time weather conditions.

In addition, the position of the front of the frame of the unmanned vehicle 11 can also be equipped with a camera module, and the captured image and the GPS coordinate image of the positioning module 14 can be returned to the background server in real time through the wireless transmission module 13, allowing the field operator to use the images on the screen to monitor the current mission execution status and set the flight path. However, the part relates to how the controlled module 12 receives mission commands through the wireless transmission module 13, and cooperates with the positioning module 14 to enable the unmanned vehicle 11 to automatically complete various tasks according to the set flight path, and the way the water quality sensor module 15 collects water is within the scope of prior art, and the composition of the details is not the main point of the present invention, so in the following description of the preferred embodiment of the present invention, all are explained together.

The management station 2 comprises a landing platform 21, a main control module 22, a wireless transmission module 23, a washing machine 24 and a charging unit 25.

The management station 2 can be a fixed or mobile base station. A platform release frame is provided on the landing platform 21, so that the main control module 22 can control the platform release frame to firmly hold the unmanned vehicle 11 before release, and when released, it can assist the unmanned vehicle 11 to lift off smoothly. But it is not limited to this, you can also track and record the flight path and movement of the unmanned vehicle 11 in real time.

In this embodiment, the washing machine 24 has a washing water tank, and an inlet pipe and a drainpipe are connected to the washing water tank, and an electric brush or a nozzle is provided inside the washing water tank. When the water quality sensor machine 1 is positioned on the landing platform 21, the sensor head of the water quality sensor module 15 will extend into the washing water tank, and each sensor head will be cleaned by the electric brush or nozzle, and then the washed water or cleaning liquid is discharged through the drainpipe, so as to realize the functions of automatic washing (brushing), water inlet, drainage, and adding chemical (such as detergent).

When the charging unit 25 supplements the power of the water quality sensor machine 1 clamped on the landing platform 21, the power battery 16 is charged by wired or wireless power transmission. After charging for a preset time, it will automatically power off, so that the water quality sensor machine 1 has enough power to travel to and from the management station 2 and will not be limited by distance and flight time.

When the present invention is used, the controlled module 12 of the water quality sensor machine 1 can obtain the task command issued by the management program of the background server or cloud processing platform through the wireless transmission module 13, so that the unmanned vehicle 11 can cooperate with the positioning module 14 according to automatically set at least two sets of flight routes to and from a sensing point 3, and after successfully taking off from the landing platform 21 of the management platform 2, the unmanned vehicle 11 can automatically fly to the designated sensing point 3 to perform various collection tasks. The preferred implementation of the sensing point 3 can be an aquaculture pond or a fish farm, but not limited to this, it can also be a natural water area (such as rivers, lakes, etc.) or artificial water bodies (such as barrage, reservoir, etc.), a positioning mark can also be set at each sensing point 3, as a foolproof positioning of the unmanned vehicle 11.

When the unmanned vehicle 11 reaches above the water surface in the designated sensing point 3, its retracting mechanism will drive the water quality sensor module 15 to swing downward, which can change the direction and angle of the water quality sensor's sensor head, or it is possible to park the unmanned vehicle 11 directly on the water surface through the internal buoyancy cabin or add a floating kit, so that the sensor head of the water quality sensor can be extended below the water surface to collect various water quality parameter data in the water. After the integration of various water quality parameter data through the controlled module 12, the wireless transmission module 13 or 23 will transmit the data back to the main control module 22 of the management station 2 or the background server for storage in real time for processing by the background server or the cloud processing platform for various monitoring or control activities. It can maneuver multiple different sensing points 3, so that field operators can use a single water quality sensor machine 1 to monitor multiple sensing points 3, so as to achieve the purpose of multi-point and time-sharing monitoring of the environment in a wide area. Thus, there is no need to install multiple sets of water quality collection devices in different water bodies that need to be monitored, which can effectively reduce the cost of construction.

After the unmanned vehicle 11 returns to the management station 2, and landed on the landing platform 21 and firmly clamped by the platform release frame, the main control module 22 will automatically control the washing machine 24 to wash each sensor head on the water quality sensor module 15 and also control the charging unit 25 to quickly charge the power battery 16. After washing and charging, the unmanned vehicle 11 continues to fly to the next sensing point 3 according to the set flight path to collect water quality parameter data. Such actions are repeated until the end of all tasks. But in practical application, it is not limited to this. You can also complete more than one task according to the flight distance and time provided by the power battery 16, and then return to the management station 2 for washing and power charging. And through the main control module 22, it can track and record the flight path and movement of the unmanned vehicle 11 in real time, so that the background server or cloud processing platform can plan the next flight route. In the maintenance of the water quality sensor module 15, it can also be automatically washed by the washing machine 24 to simplify manpower and reduce maintenance costs.

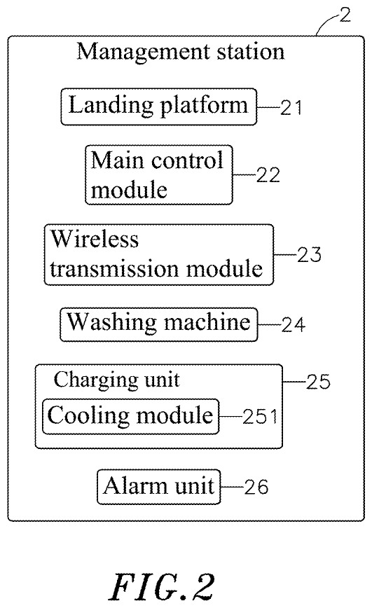

Referring also to FIG. 2, in an alternate form of the present invention, the charging unit 25 of the management station 2 is equipped with a cooling module 251. The cooling module 251 is preferably implemented as a cooling fan, but it is not limited to this, and the cooling fan can be further equipped with a water-cooled or air-cooled radiator to form a circulating cooling system. The cooling module 251 can be used to directly cool the water quality sensor machine 1 or the power battery 16 to avoid the loss of the high temperature of the lithium battery of the power battery 16 during the rapid charging process or the reduction of charging performance. In turn, the overall service life is extended, and the charging operation is safer and more stable.

In this alternate form, the main control module 22 of the management station 2 can manage the water quality sensor machine 1, including the rise and fall of the unmanned vehicle 11, real-time tracking and recording the flight trajectory, etc. The management station 2 in accordance with this alternate form further comprises an alarm unit 26 electrically connected to the main control module 22. When the main control module 22 detects abnormal conditions in the unmanned vehicle 11, such as failure to connect or disconnect, insufficient power, or malfunction, etc., the alarm unit 26 will issue an alarm signal externally, including the sound, voice, light sources or electronic messages (such as e-mail, text messages, or broadcast notification services), etc., to inform the on-site operator to take immediate actions, ensuring the normal operation of the unmanned vehicle 11.

Although particular embodiments of the invention have been described in detail for purposes of illustration, various modifications and enhancements may be made without departing from the spirit and scope of the invention. Accordingly, the invention is not to be limited except as by the appended claims.

What the invention claimed is:

1. A management station of multi-point time-sharing water quality monitoring system for managing at least one water quality sensor machine, each said water quality sensor machine comprising an unmanned vehicle and a water quality sensor module carried on said unmanned vehicle, said unmanned vehicle being adapted to receive a task command to travel to and from a number of designated sensing points according to a pre-set flight path and to perform various water quality parameter data collection tasks through the said water quality sensor module, a management station comprising a landing platform for the positioning of said unmanned vehicle, a main control module, a wireless transmission module, a washing machine and a charging unit, said main control module being adapted to manage the operation of said water quality sensor machine and receive said various water quality parameter data through said wireless transmission module, wherein when said unmanned vehicle returns to said landing platform from one said sensing point, said main control module automatically controls said washing machine to wash said water quality sensor module, and also controls said charging unit to charge a power battery installed on said unmanned vehicle.

2. The management station of multi-point time-sharing water quality monitoring system as claimed in claim 1, wherein said landing platform comprises a platform release frame, said platform release frame being controlled by said main control module to firmly hold said unmanned vehicle for letting said unmanned vehicle to be washed by said washing machine and charged by said charging unit before release, and to assist said unmanned vehicle to lift off when released.

3. The management station of multi-point time-sharing water quality monitoring system as claimed in claim 1, wherein said washing machine comprises a washing water tank into which a sensing head of said water quality sensor module extends, and an inlet pipe and a drainpipe connected to said washing water tank.

4. The management station of multi-point time-sharing water quality monitoring system as claimed in claim 3, wherein said washing machine further comprises an electric brush and/or a nozzle provided inside said washing water tank for brushing or washing said sensing head of said water quality sensor module.

5. The management station of multi-point time-sharing water quality monitoring system as claimed in claim 1, wherein said charging unit is used to charge said power battery of said water quality sensor machine by wired or wireless power transmission selectively.

6. The management station of multi-point time-sharing water quality monitoring system as claimed in claim 1, wherein said charging unit comprises a cooling module for cooling said power battery of said water quality sensor machine.

7. The management station of multi-point time-sharing water quality monitoring system as claimed in claim 6, wherein said cooling module is a cooling fan.

8. The management station of multi-point time-sharing water quality monitoring system as claimed in claim 1, further comprising an alarm unit electrically connected to said main control module, said alarm unit sending out an alarm signal to the outside when said main control module detects abnormal conditions of said unmanned vehicle, said alarm signal including warning sound, voice, light source or electronic message.
